(19) United States
(12) Patent Application Publication
(10) Pub. No.: US 2025/0032768 A1
(43) Pub. Date: Jan. 30, 2025

(54) PORT ASSEMBLIES

(71) Applicant: B. Braun Medical Inc., Bethlehem, PA (US)

(72) Inventors: Elisha James Endres, Bethlehem, PA (US); David Sanders, Bethlehem, PA (US)

(73) Assignee: B. Braun Medical Inc., Bethlehem, PA (US)

(21) Appl. No.: 18/225,814

(22) Filed: Jul. 25, 2023

(51) Int. Cl. A61M 39/10

(52) U.S. Cl. CPC A61M 39/10; A61M 2039/1022

(57) ABSTRACT

Systems and devices for replacing connectors using a modular port assembly. An infusion pump assembly includes a main assembly with an opening that is surrounded by a bezel. The bezel is sized and shaped to releasably receive a port assembly. The port assembly includes a connector that is oriented to releasably engage with a receptacle located within the main assembly and near the opening, and thereby form an operable connection between the connector and its receptacle (e.g., using a cable). The assembly optionally includes a gasket that is sized and shaped to rest between an edge of the port assembly and the bezel. The port assembly is releasably secured with one or more fasteners.

### TECHNICAL FIELD

Examples set forth in the present disclosure relate to the field of medical devices, such as devices for delivering a beneficial agent or fluid to a patient. More particularly, but not by way of limitation, the present disclosure describes a replaceable port assembly with one or more connectors for attaching external devices to a medical device, such as an infusion pump.

### BACKGROUND

Infusion pumps deliver controlled doses of beneficial agents in fluid form, such as medications, analgesics, and nutrition to patients. Infusion pumps are particularly well suited to delivering controlled doses of fluids from a bottle or bag, through flexible tubing, and into a patient over a relatively long period (e.g., several hours or longer). Various types of fluid delivery systems are in use, including vacuum-driven systems and devices that use a mechanical pump. For example, a peristaltic infusion pump includes a rotor assembly or other element that exerts a force on the flexible tubing, thereby driving the fluid into the patient at a controlled rate.

### BRIEF DESCRIPTION OF THE DRAWINGS

Features of the various examples described will be readily understood from the following detailed description, in which reference is made to the figures. A reference numeral is used with each element in the description and throughout the several views of the drawing. When a plurality of similar elements is present, a single reference numeral may be assigned to like elements, with an added upper or lower-case letter referring to a specific element.

The various elements shown in the figures are not drawn to scale unless otherwise indicated. The dimensions of the various elements may be enlarged or reduced in the interest of clarity. The several figures depict one or more implementations and are presented by way of example only and should not be construed as limiting. Included in the drawing are the following figures:

FIG. 1 is a perspective, exploded view of an example infusion pump assembly including a main assembly and a port assembly aligned with an opening in the main assembly; and

FIG. 2 is a side, exploded view of an example port assembly with connectors, each of which is configured to facilitate access for connection and disconnection with a receptacle inside the main assembly, using a cable or other intermediate connector.

### DETAILED DESCRIPTION

Various implementations and details are described with reference to example port assemblies for use with infusion pumps. In an example implementation, an infusion pump assembly includes a main assembly that has an opening surrounded by a bezel. The bezel is sized and shaped to releasably receive a port assembly. The port assembly includes a connector that is oriented to releasably engage with a receptacle located within the main assembly and near the opening, and thereby form an operable connection between the connector and its receptacle. The assembly optionally includes a gasket that is sized and shaped to rest between an edge of the port assembly and the bezel. The port assembly is releasably secured with one or more fasteners. The example port assembly in some implementations includes one or more additional connectors, each of which is sized, shaped, and oriented to releasably engage with a corresponding additional receptacle located within the main assembly using, for example, a cable or other intermediate connector. When any component of the port assembly is faulty, a new port assembly is installed or the fault component is replaced, without the need to disassemble the main assembly to restore the operable connections.

The following detailed description includes assemblies, systems, methods, and techniques to illustrate the examples set forth in the disclosure. Numerous details and examples are included for the purpose of providing a thorough understanding of the disclosed subject matter and its relevant teachings. Those skilled in the relevant art, however, may understand how to apply the relevant teachings without such details. Aspects of the disclosed subject matter are not limited to the specific devices, systems, and methods described because the relevant teachings can be applied or practiced in a variety of ways. The terminology and nomenclature used herein is for the purpose of describing particular aspects only and is not intended to be limiting. In general, well-known instruction instances, protocols, structures, and techniques are not necessarily shown in detail.

The term “connect,” “connected,” “couple,” and “coupled” as used herein refers to any logical, optical, physical, or electrical connection, including a link or the like by which the electrical or magnetic signals produced or supplied by one system element are imparted to another coupled or connected system element. Unless described otherwise, coupled, or connected elements or devices are not necessarily directly connected to one another and may be separated by intermediate components, elements, or communication media, one or more of which may modify, manipulate, or carry the electrical signals. The term “on” means directly supported by an element or indirectly supported by the element through another element integrated into or supported by the element.

The term “proximal” or “proximate” is used to describe an item or part of an item that is situated near, adjacent, or next to an object or person; or that is closer relative to other parts of the item or part, which may be described as “distal.” For example, the end of an item nearest an object may be referred to as the proximal end, whereas the generally opposing end may be referred to as the distal end.

The term “facilitate,” as used herein, means to aid, assist, enable, improve, or make easier. The term “inhibit,” as used herein, means to hinder, restrain, impede, thwart, oppose, obstruct, or interfere with.

The orientations of the main pump housing, port assembly, connectors, receptacles, fasteners, and associated components as shown in any of the drawings are given by way of example only, for illustration and discussion purposes. For example, in operation, a main assembly may be oriented as shown or in any other orientation that is suitable to a particular application. Also, to the extent used herein, any directional term, such as front, rear, inwards, outwards, towards, left, right, lateral, longitudinal, up, down, upper, lower, top, bottom and side, are used by way of example only, and are not limiting as to direction or orientation of any component described herein.

Additional objects, advantages and novel features of the examples will be set forth in part in the following description, and in part will become apparent to those skilled in the art upon examination of the following and the accompanying drawings or may be learned by production or operation of the examples. The objects and advantages of the present subject matter may be realized and attained by means of the methodologies, instrumentalities and combinations particularly pointed out in the appended claims.

The systems, devices, and methods described herein can be used for any of a variety of ports contained within a port assembly that facilitate an operable connection between an external device (e.g., an external electronic device or any other external device) and an internal component (e.g., a computer, a microcontroller, a processor, or any other internal element) within the main assembly of a device (e.g., an infusion pump).

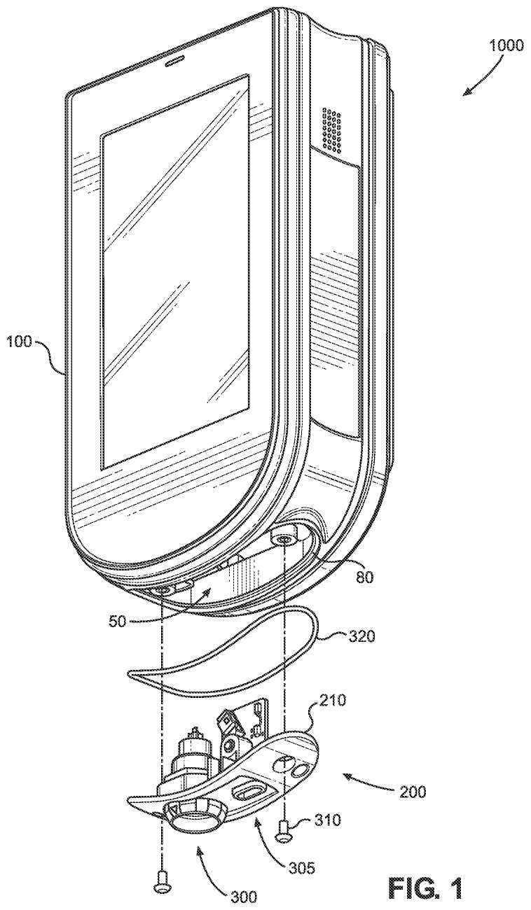

FIG. 1 is a perspective, exploded view of an example infusion pump assembly 1000 including a main assembly 100 and a port assembly 200. The main assembly 100 includes an opening 50 (e.g., along the bottom surface, in this view). The opening 50 is at least partly surrounded by a bezel 80. The term “bezel,” as used herein, means and includes a groove or other feature along the edge of an opening which is shaped to engage with and hold an object, such as a cover, when the object is inserted or otherwise placed against the opening. For example, the raised ring or flange around a watch face typically includes a bezel that is shaped to engage with a watch crystal and retain it (e.g., creating an interference fit between the watch crystal and the bezel). As shown in FIG. 1, the bezel 80 in some implementations is sized and shaped to releasably receive the port assembly 200.

The port assembly 200 in some implementations includes a connector 300 and one or more additional connectors 305, and utilizes one or more fasteners 310 (e.g., a pair of bolts or screws aligned with corresponding holes, as shown) for releasably securing the port assembly 200 to the main assembly 100.

As shown in FIG. 1, the port assembly 200 in some implementations includes a gasket 320 that is sized and shaped to rest between an edge 210 of the port assembly 200 and the bezel 80. The gasket 320 in some implementations rests in a groove around the opening 50 and thus provides a seal between the bezel 80 and the inside face of the port assembly 200. In some implementations, the edge 210 of the port assembly 200 is sized and shaped to engage with the bezel 80 without a gasket 320 therebetween.

The gasket 320 in some implementations is releasably attached to all or part of the edge 210 or, in other implementations, to all or part of the bezel 80. The gasket 320 in some implementations is not attached to any other element or component. In use, when the port assembly 200 is connected to the main assembly 100, the gasket 320 at least partly fills the space between the edge 210 of the port assembly 200 and the bezel 80, facilitating a seal that inhibits the intrusion or leakage of air, water, or other elements.

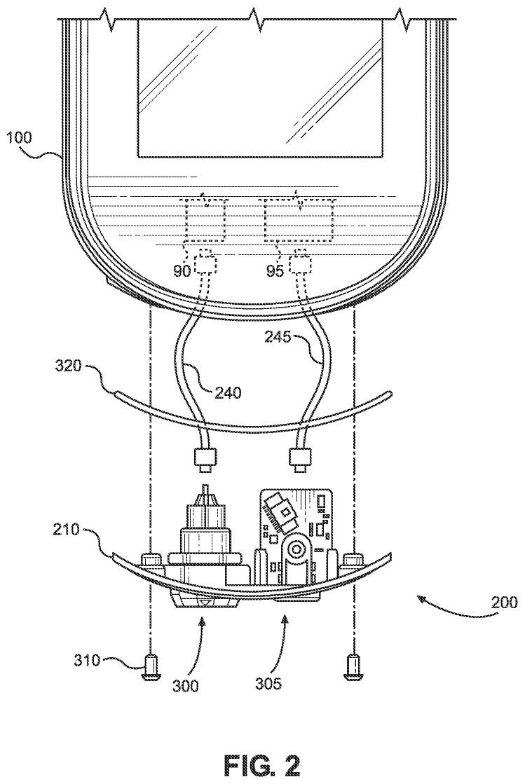

FIG. 2 is a side, exploded view of an example port assembly 200 with connectors 300, 305, each of which connects with its associated receptacle 90, 95 located inside the main assembly 100.

As shown in FIG. 2, a connector 300 is sized and shaped, and oriented on the port assembly 200, so that it will releasably connect and disconnect with a corresponding receptacle 90 located within the main assembly 100. The connector 300 in some implementations is a bolus accessory connector, by and through which an external device (e.g., an external electronic device) (not shown) is connected to an internal receptacle 90 associated with internal component (e.g., a microcontroller or processor, or any other internal element). In use, the connection between the connector 300 of the port assembly 200 and the receptacle 90 of the main assembly 100 creates or otherwise facilitates an operable connection for an external device (e.g., an electrical connection between the pump assembly and an external device to facilitate or otherwise control the pumping of a bolus of fluid). In the example shown, the connector 300 connects to the receptacle 90 using a cable 240.

Similarly, as shown, an additional connector 305 is sized and shaped, and oriented on the port assembly 200, so that it will releasably connect and disconnect with a corresponding additional receptacle 95 located within the main assembly 100. The additional connector 305 in some implementations is an electronic port (e.g., a USB port,) by and through which an external device (e.g., a power source, a computer, or any other external electronic device) is connected to an internal additional receptacle 95 associated with internal component (e.g., a microcontroller or processor, or any other internal element). In use, the connection between the additional connector 305 on port assembly 200 and the additional receptacle 95 on the main assembly 100 creates or otherwise facilitates an additional operable connection (e.g., a wired electronic connection between devices) between the additional connector 305 and the additional receptacle 95. The USB port in some implementations includes or is part of a printed circuit board assembly (PCBA) located on the interior surface of the port assembly 200, as shown in FIG. 2. In this example, the additional operable connection may include a wired connection (e.g., in which the PCBA is connected to the additional receptacle 95). In the example shown, the additional connector 305 connects to the additional receptacle 95 using an additional cable 245.

In use, when the port assembly 200 is unsecured or otherwise disconnected from the main assembly 100, the removal facilitates access to both the operable connection between the connector 300 and the receptacle 90 (e.g., including cable 240) and the additional operable connection between the additional connector 305 and the additional receptacle 95 (e.g., including the additional cable 245) without the need to disassemble the main assembly 100. In this aspect, each connector 300, 305 is shaped and sized (e.g., sized in height relative to the port assembly 200) and oriented so that it will allow access for connection and disconnection with its respective receptacle 90, 95 when the port assembly 200 is unsecured (e.g., unsecured by removing the one or more fasteners 310 and pulling the port assembly 200 away from the main assembly 100).

During removal, in some implementations, each connector 300, 305 includes a cable 240, 245 having sufficient length to allow the port assembly 200 to be pulled away from the main assembly 100. The example cables 240, 245 shown in FIG. 2 are not engaged with their respective connectors 300, 305 or receptacles 90, 95. In some implementations, each cable 240, 245 includes a plug or socket on each end that is sized and shaped to engage with a corresponding plug or socket on its respective connector 300, 305 and receptacle 90, 95. The cables 240, 245 in some implementations are readily connected or disconnected without the use of a tool. In some implementations, each cable 240, 245 is sized in length (e.g., with additional or excess length, to create a desired amount of slack) to allow separation of the port assembly 200 without causing an inadvertent disconnect of the operable connections between each connector 300, 305 and its receptable 90, 95. In this aspect, when the port assembly 200 is separated from the main assembly 100, one or both cables 240, 245 may be selectively disconnected. For example, if replacing the connector 300, the cable 240 may be disconnected from the receptacle 90 without disconnecting the additional cable 245. If replacing both connectors 300, 305, each cables 240, 245 may be disconnected from its respective receptacle 90, 95 during the removal and replacement process.

In operation, one or more operable connections (e.g., between connector and receptacle) may fail or otherwise become inoperable. For example, a failure may occur in one or both connectors 300, 305. In some implementations, in response to detecting a faulty connection, the existing port assembly 200 is removed—with or without determining which part of element is faulty. The engagement of a new or subsequent port assembly 200 to the main assembly 100 facilitates a replacement of both operable connections. In this aspect, such a replacement can be accomplished by relatively untrained or non-technical personnel, without the need to use any tools or equipment to detect, isolate, or otherwise diagnose which part or element is faulty—without the need to disassemble the main assembly 100—thereby saving time and expense while facilitating a prompt return to normal operations.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as plus or minus ten percent from the stated amount or range.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An infusion pump assembly comprising:
a main assembly defining an opening surrounded by a bezel sized and shaped to releasably receive a port assembly, the port assembly comprising:
a connector oriented to releasably engage with a receptacle within the main assembly; and
one or more fasteners for releasably securing the port assembly to the main assembly.

2. The infusion pump assembly of claim 1, wherein the port assembly further comprises:
a gasket sized and shaped to rest between an edge of the port assembly and the bezel.

3. The infusion pump assembly of claim 2, wherein the gasket is releasably attached to a portion of at least one of the edge and the bezel.

4. The infusion pump assembly of claim 1, wherein the releasable securing between the port assembly and the main assembly facilitates access to an operable connection between the connector and the receptacle.

5. The infusion pump assembly of claim 4, wherein the connector comprises a bolus accessory connector and the receptacle is operably connected to an internal component supported within the main assembly.

6. The infusion pump assembly of claim 1, wherein the port assembly further comprises:
an additional connector oriented to releasably engage with an additional receptacle within the main assembly.

7. The infusion pump assembly of claim 6, wherein the additional connector comprises an electronic port and the additional receptacle is operably connected to an additional internal component supported within the main assembly.

8. The infusion pump assembly of claim 6, wherein the releasable securing between the port assembly and the main assembly facilitates:
a disconnect of the port assembly from the main assembly;
access to an operable connection between the connector and the receptacle; and
access to an additional operable connection between the additional connector and the additional receptacle, such that the main assembly does not require disassembly to access the operable connection.

9. The infusion pump assembly of claim 8, further comprising:
a cable between the connector and the receptacle which is sized in length to facilitate the disconnect while maintaining the operable connection; and
an additional cable between the additional connector and the additional receptacle which is sized in length to facilitate the disconnect while maintaining the additional operable connection.

10. The infusion pump assembly of claim 8, wherein at least one of the operable connections is faulty, and
wherein engagement of a subsequent port assembly to the main assembly facilitates a replacement of both operable connections.

11. A port assembly comprising:
a connector oriented to releasably engage with a receptacle located within a main assembly, wherein the main assembly defines an opening surrounded by a bezel, and wherein the opening is sized and shaped to releasable receive the port assembly; and
one or more fasteners for releasably securing the port assembly to the main assembly.

12. The port assembly of claim 11, further comprising:
a gasket sized and shaped to rest between an edge of the port assembly and the bezel.

13. The port assembly of claim 12, wherein the gasket is releasably attached to a portion of at least one of the edge and the bezel.

14. The port assembly of claim 11, wherein the releasable securing between the port assembly and the main assembly facilitates access to an operable connection between the connector and the receptacle.

15. The port assembly of claim 11, wherein the connector comprises a bolus accessory connector and the receptacle is operably connected to an internal component supported within the main assembly.

16. The port assembly of claim 11, further comprising:
an additional connector oriented to releasably engage with an additional receptacle within the main assembly.

17. The port assembly of claim 16, wherein the additional connector comprises an electronic port and the additional receptacle is operably connected to an additional internal component supported within the main assembly.

18. The port assembly of claim 16, wherein the releasable securing between the port assembly and the main assembly facilitates:
a disconnect of the port assembly from the main assembly;
access to an operable connection between the connector and the receptacle; and
access to an additional operable connection between the additional connector and the additional receptacle, such that the main assembly does not require disassembly to access the operable connection.

19. The port assembly of claim 18, further comprising:
a cable between the connector and the receptacle which is sized in length to facilitate the disconnect while maintaining the operable connection; and
an additional cable between the additional connector and the additional receptacle which is sized in length to facilitate the disconnect while maintaining the additional operable connection.

20. The port assembly of claim 18, wherein at least one of the operable connections is faulty, and
wherein engagement of a subsequent port assembly to the main assembly facilitates a replacement of both operable connections.
